(19) United States
(12) Patent Application Publication
(10) Pub. No.: US 2012/0301923 A1
(43) Pub. Date: Nov. 29, 2012

(54) DIALYSIS FERMENTER-BIOREACTOR WITH DIALYSIS DEVICE

(75) Inventors: Artur Hoffmann, Wolfratshausen (DE); Max Lechner, Penzberg (DE); Christoph Reese, Muenchen (DE)

(73) Assignee: ROCHE DIAGNOSTICS OPERATIONS, INC., Indianapolis, IN (US)

(21) Appl. No.: 13/480,446

(22) Filed: May 24, 2012

(30) Foreign Application Priority Data
May 27, 2011 (EP) 11167948.6

(51) Int. Cl. C12M 1/12; C12N 5/12; C12N 5/10; C12P 21/00; C12N 9/00; C12N 5/07; C12N 5/071

(52) U.S. Cl. 435/70.21; 435/297.4; 435/348; 435/346; 435/326; 435/394; 435/70.1; 435/183

(57) ABSTRACT

The present disclosure provides systems and methods of using a semipermeable membrane in a dialysis fermenter as a separation layer between a cell-containing liquid culture medium and a non-cell-containing dialysis medium. In some embodiments, the semipermeable membrane may have a molecular cut-off of 15 kDa to 50 kDa. The instant disclosure also provides a dialysis fermenter with compartments for cell-containing culture medium, non-cell-containing nutrient solution as well as an exchange unit having a semipermeable membrane, wherein mass transfer takes place between the culture medium and the dialysis medium by means of diffusion and/or ultrafiltration. Methods for culturing cells are also disclosed.

### PRIORITY CLAIM

This application claims the benefit of European Patent Application No. 11167948.6, filed May 27, 2011, the disclosure of which is hereby incorporated by reference in its entirety.

### FIELD OF THE DISCLOSURE

The present disclosure relates to bioreactors. More specifically, the present disclosure relates to bioreactors, such as fermenters, capable of culturing biological cells.

### BACKGROUND

In dialysis membranes may act as a molecular sieve which has a certain cut-off sometimes specified as the largest molecular weight of particles which can still pass the membrane during dialysis. Accordingly, molecules having a molecular weight below the cut-off can diffuse through the membrane, whereas larger molecules having a molecular weight above the cut-off are retained by the membrane (i.e., they cannot permeate through the membrane). In practical applications the cut-off must be suitably selected so that essential high molecular weight ingredients (e.g., growth factors) are not removed from the culture medium (which cells may be suspended in) into the nutrient solution during dialysis. This would have undesired consequences for cell growth. Since the compositions of cell culture media are often complex especially in the case of mammalian cells, a low cut-off may be advantageous.

Filter modules consisting of Cuprophan® hollow fibres (Membrana GmbH, Wuppertal, Germany) are in the lower range of the cut-off described in previously published documents (for example, Cuprophan® RC55 8/200 hydrophilic capillary membrane has a wall thickness of 8.4 μm±1.1 μm, an inner diameter of 200 μm±15 μm, and a cut-off of about 12 kDa molecular weight). Properties of Cuprophan hollow fibres may be found in the manufacturer data sheet 456/9095/000 (02/00).

When Cuprophan®-based devices are used for blood dialysis (e.g., haemodialyzers) however, physiological reactions of blood cells with the membrane material have been shown to occur (see for example, Brandt T. and Wiese F., Contrib. Nephrol. 138 (2003) 1-12; Daveport A., Hemodial. Int. 12 Suppl. 2 (2008) p 29-p33, and http://www.dialysemuseum.de/prinzip_membranen.php published Mar. 17, 2011). For this reason filter modules consisting of Cuprophan® hollow fibres are no longer available on the market even for dialysis fermentation. Thus, alternative membrane materials for dialysis fermentation must be identified having improved properties.

### SUMMARY OF THE DISCLOSURE

According to embodiments of the instant disclosure and as disclosed herein, it was surprisingly found that membranes with a higher cut-off than that of Cuprophan® have a comparably good or improved effect in dialysis fermentation. Whereas Cuprophan® has a relatively closed surface, it was additionally surprisingly found that materials with larger pores could be advantageously used as a dialysis membrane. Even more surprising was the fact that these advantages were also observed for hollow fibres having a more than 3-fold larger membrane thickness (thickness of the wall formed by the membrane defined as the average shortest path which in the present case separates the compartments of the culture medium and of the dialysis medium) compared to Cuprophan® hollow fibres (8 μm membrane thickness).

According to embodiments of the subject disclosure, the use of a semipermeable membrane having a molecular cut-off of 15 kDa to 50 kDa in a dialysis fermenter as a separation layer between (a) the cell-containing liquid culture medium, and (b) the non-cell-containing dialysis medium (nutrient solution) is disclosed. Furthermore embodiments of the instant disclosure provide a dialysis fermenter with compartments for cell-containing culture medium, non-cell-containing nutrient solution as well as an exchange unit of a semipermeable membrane, wherein mass transfer takes place between the culture medium and the dialysis medium by means of diffusion and/or ultrafiltration. Furthermore, embodiments of the instant disclosure also provide a method for culturing cells.

Embodiments of the instant disclosure comprise bioreactors, which comprise devices for culturing biological cells, in some embodiments on an industrial scale. According to embodiments of the disclosure, an improved dialysis fermenter (e.g., bioreactor) comprising a first and a second compartment is disclosed. In some embodiments, culture medium and biological cells are located in a first compartment and nutrient solution is provided in a second compartment. According to some embodiments, the operation of the bioreactor (e.g., fermentation process) propagates and/or cultures the cells and the cells produce desired products in this process and secrete these products if necessary into the surrounding culture medium. Further, embodiments of the instant disclosure allow mass transfer to occur during the fermentation process between the liquid phases in the first and second compartment by means of diffusion through a semipermeable membrane. Such mass transfer is referred to as dialysis and plays a role in enabling the transport of undesired metabolic products out of the culture medium. In addition, consumed nutrients of the culture medium may be replenished from the nutrient solution (dialysis medium) by dialysis. Embodiments of the present disclosure also provide other particular advantageous properties for certain semipermeable membranes in dialysis which will become apparent throughout the disclosure.

Some embodiments of the disclosure include the use of a semipermeable membrane having a molecular cut-off of more than 15 kDa to 50 kDA in a dialysis fermenter as a separation layer between (a) the cell-containing liquid culture medium, and (b) the non-cell-containing dialysis medium (nutrient solution).

Some embodiments of the disclosure provide a dialysis fermenter comprising a first compartment (adapted for containing a cell-containing liquid culture medium) and a second compartment (adapted for containing a non-cell-containing dialysis medium, for example a nutrient solution), and additionally comprising an exchange unit (for example, dialyzer) with a semipermeable membrane (adapted for dipping into the culture medium in the first compartment for example), wherein the exchange unit (e.g., filter module) is fluidically connected to the second compartment by an inlet as well as an outlet, further comprising a pump (adapted for feeding dialysis medium from the second compartment into the exchange unit and from there back again into the second compartment), wherein mass transfer may take place between the culture medium and the dialysis medium along the semipermeable membrane by means of diffusion and/or ultrafiltration. In some embodiments, the molecular cut-off of the semi-permeable membrane is in a range of about 15 kDa to about 50 kDa, where 15 kDa and 50 kDa are included in the range.

According to additional embodiments of the instant disclosure, a process for culturing cells is provided. According to some embodiments, a cell culture is kept in substantially homogeneous suspension in a culture medium by means of a stirring device under controlled environmental conditions in a biorector (e.g., a dialysis fermenter). In exemplary embodiments, nutrients for the cells are fed in and waste products of the cells are discharged, wherein a dialysis medium (e.g., nutrient solution) that is separate from the culture medium is used which flows in a flow path which is separated from the culture medium by a semipermeable membrane where the membrane is designed such that it is permeable to the nutrients and the waste products of the cells but is impermeable to higher molecular components of the culture medium and wherein the culture medium containing the cells is led past one side of the membrane and the dialysis medium containing the nutrients is led past the other side of the membrane such that nutrients from the dialysis medium can pass through the membrane into the culture medium and waste products from the culture medium can pass into the dialysis medium. In some embodiments, the molecular cut-off of the membrane is in the range of about 15 kDa to about 50 kDa, where 15 kDa and 50 kDa are included in the range.

The above-described embodiments of the various aspects of the disclosure may be used alone or in any combination thereof without departing from the scope of the disclosure.

### BRIEF DESCRIPTION OF THE DRAWINGS

The features and advantages of this disclosure, and manner of attaining them, will become more apparent and disclosure itself will be better understood by reference to the following description of embodiments of the disclosure taken in conjunction with the accompanying drawings, which are incorporated in and constitute a part of the specification. In the drawings, each figure contains two diagrams denoted “A” and “B”. Each diagram denoted “A” represents data from fermentation 1 using a PES module according to the instant disclosure having a filter located upstream. Each diagram denoted “B” represents data from fermentation 2 using a PES module according to the instant disclosure without a corresponding filter. In each diagram the X axis denotes the time in days. The Y axis is divided into relative units in each diagram. In corresponding diagrams “A” and “B” equal relative values also denote equal absolute measured values. If for example a relative value of 0.6 for the parameter live cell density is stated in the respective diagram for fermentation 1 and fermentation 2 this corresponds in both cases to an absolute measured value determined to be of equal magnitude. The same applies to other parameters listed in the following.

FIGS. 1a and 1b are line graphs showing a Live cell density (cells per volume) in culture medium;

FIGS. 2a and 2b are line graphs showing product concentration in the culture medium of monoclonal antibody secreted by hybridoma cells;

FIGS. 3a and 3b are line graphs showing a module input pressure (i.e., the pressure in the feed line to the exchange unit generated by the pump which conveys dialysis medium from the second compartment into the exchange unit and from there back again into the second compartment);

FIGS. 4a and 4b are line graphs showing a dialysis flow (i.e., the volume of dialysis medium which is pumped per unit of time) through the exchange unit; and

FIGS. 5a and 5b are line graphs showing lactate concentration (the solid line denotes the measure values in the culture medium and the line interrupted by white squares denotes the measured values in the dialysis medium).

Corresponding reference characters indicate corresponding parts throughout the several views. Although the drawings represent embodiments of the present disclosure, the drawings are not necessarily to scale and certain features may be exaggerated in order to better illustrate and explain the present disclosure. The exemplifications set out herein illustrate an exemplary embodiment of the disclosure, in one form, and such exemplifications are not to be construed as limiting the scope of the disclosure in any manner.

### DETAILED DESCRIPTION OF THE EMBODIMENTS OF THE DISCLOSURE

The embodiments disclosed herein are not intended to be exhaustive or limit the disclosure to the precise form disclosed in the following detailed description. Rather, the embodiments are chosen and described so that others skilled in the art may utilize their teachings.

As used in conjunction with the present disclosure, semipermeability is the property of a substantial or physical interface. A membrane and in particular a dialysis membrane is a possible embodiment of such an interface. Another embodiment is for example a porous wall. In this connection semipermeability means that only molecules below a certain mole mass or particles below a certain size can migrate through the interface. An interface is permeable for substances which are small enough to migrate through the interface. The permeability of a substance is determined by the cut-off, a property of the interface.

In general it is desirable for the purposes of dialysis fermentation to use semipermeable membranes having cut-offs that are as low as possible. Although 100 kDa is regarded in this connection as a practical upper limit, it can be expected that important substances for the growth of the cell culture such as growth factors will pass into the dialysis medium during the dialysis process and thus leave the culture medium and be no longer available to the cells. Another problem arises in the case of products which are secreted by the cultured cells into the culture medium. During dialysis fermentation it is generally undesired that these products pass into the nutrient solution. At a cut-off of 100 kDa this, however, cannot be excluded in many cases. For this and other reasons, membranes having the lowest possible cut-off have been used up to now.

As disclosed herein, the instant disclosure provides the surprising and unexpected discovery that membranes having a relatively high cut-off (more than 12 kDa) have an equally good (or even better) effect in dialysis fermentation compared to membranes having lower-range of cut-offs (such as Cuprophan®, for example). Accordingly, some embodiments of the instant disclosure use a semipermeable membrane having a molecular cut-off of more than 15 kDa to 50 kDa (where 15 kDa and 50 kDa are included in the range) in a dialysis fermenter as a separation layer between (a) the cell-containing liquid culture medium and (b) the non-cell-containing dialysis medium.

According to the instant disclosure, suitable dialysis membranes can consist of various materials known to a person skilled in the art. For example, membranes may consist of regenerated or modified cellulose or of synthetic materials. The latter include polysulfone (PSU), polyacrylo-nitrile (PAN), polymethylmethacrylate (PMMA), mixtures of polyarylether-sulfones, polyvinylpyrrolidone and polyamide (Polyamix®) and others, for example. The polysulfones include polyethersulfone [poly(oxy-1,4-phenylsulfonyl-1,4-phenyl), abbreviated PES], for example. In some exemplary embodiments, polyethersulfone may be utilized as a semipermeable membrane for the use according to the disclosure. In some cases, according to the instant disclosure, PES membranes include increased hydrophilicity (and/or the improved wettability of the membrane with water) compared to PSU membranes. In some embodiments, the wettability of PES membranes can, for example, be further increased by the inclusion of the water-soluble polymer polyvinylpyrrolidone.

In some exemplary embodiments, the dialysis fermenter according to the disclosure comprises a first compartment containing a cell-containing liquid culture medium and a second compartment containing a non-cell-containing dialysis medium. In addition, the dialysis fermenter of some exemplary embodiments comprises an exchange unit dipping into the culture medium which is also referred to as a dialyzer. The exchange unit comprises a semipermeable membrane and the exchange unit is fluidically connected to the second compartment by an inlet as well as an outlet. A further component is a pump which conveys dialysis medium from the second compartment into the exchange unit and from there back again into the second compartment. In this process mass transfer between the culture medium and the dialysis medium occurs along the semipermeable membrane in the first compartment by means of diffusion and/or ultrafiltration. The dialysis fermenter according to some exemplary embodiments of the instant disclosure comprises a molecular cut-off of the aforementioned semipermeable membrane in the range of 15 kDa to 50 kDa, where 15 kDa and 50 kDa are included in the range.

In some exemplary embodiments of the instant disclosure the molecular cut-off of the semipermeable membrane is in the range of 20 kDa to 40 kDa, where 25 kDa and 40 kDa are included in the range. Further exemplary embodiments may comprise a cut-off of 35 kDa (±5%), for example.

According to the instant disclosure, the permeability of a membrane is inversely proportional to the thickness of this membrane (where the thickness of the membrane corresponds to the average shortest distance by which the membrane separates the liquid phases of the culture medium and the nutrient solution in the dialyzer). According to some exemplified embodiments of the instant disclosure, membranes for the use in a dialysis fermenter disclosed herein are characterized by a thickness of about 15 μm to 50 μm. In some specific embodiments the thickness is about 35 μm, for example.

According to embodiments of the instant disclosure, suitable materials from which the membranes may be manufactured include polyethersulfone, polysulfone, polyacrylonitrile (PAN), polymethylmethacrylate (PMMA), mixtures of polyarylethersulfone, polyvinylpyrrolidone and polyamide (Polymix®), and other suitable materials known to a person skilled in the art.

The membranes can for example be integrated in the dialyzer as lamellae. However, other forms are also possible. In some embodiments the membranes may be present as hollow fibres. A hollow fibre is understood as a cylindrical fibre having one or more continuous hollow spaces in the cross-section. Hollow fibres (hollow fibre synonymous with capillary membrane) may be manufactured with semipermeable structures so that the walls of the fibre act as a membrane. The inner diameter of some embodiments of hollow fibres according to the instant disclosure may be 50 μm to 500 μm and their outer diameter may be 65 μm to 600 μm. In some exemplified embodiments, the outer diameter of the hollow fibres may be 260 μm±10% and the inner diameter may be 190 μm±10%.

Construction of filter modules, according to the instant disclosure, may comprise hollow fibres having a typical length between 10 cm and more than 100 cm being combined to form modules having a large filter surface, and the hollow fibres may be sealed at both ends against hydraulic short circuit. This arrangement, according to the instant disclosure, comprises a particular embodiment for practicing the teachings presented herein. In a exemplified embodiment, the exchange unit may have a cylindrical shape. The degree of filling of the exchange unit may also be determined by the ratio of the cross-sectional area occupied by hollow fibres to the total cross-sectional area of the cylinder on which the hollow fibres are distributed. In some such embodiments, the degree of filling in the exchange unit may be 0.19 to 0.50. It was discovered herein that values within this range assist in providing a good diffusion of the exchange unit with culture medium and the exchange area of the hollow fibres may be, as disclosed herein, within an advantageous range for such embodiments. According some exemplified embodiments, the degree of filling comprises a value in the range of about 0.30 to 0.42. In some embodiments, a value of about 0.35 for the degree of filling is provided.

According to some exemplified embodiments of the exchange unit disclosed herein, hollow fibres may be equally distributed over the cross-sectional area of this exchange unit and arranged as a bundle that is not further subdivided (as opposed to an arrangement of hollow fibres in fibre bundles that are separated from one another).

In a specific embodiment the cells may be cultured as a suspension in the first compartment of the dialysis fermenter. This means that the cells usually do not adhere to surfaces but are basically uniformly distributed in the culture medium (homogeneous suspension). A stirring unit extending into the first compartment may be used for providing a uniform distribution of the cells during operation.

Embodiments of the subject disclosure include the hollow fibres of the exchange unit placed in the culture medium of the first compartment comprising an additional holder or bundling, enabling for removal or replacement of the hollow fibres in an uncomplicated manner (although in some embodiments of the present disclosure it is possible that the hollow fibres may be placed in the culture medium without a holder). According to embodiments comprising a holder, the holder aides the hollow fibres when subject to continuous and sometimes strong mechanical loads due to the movement of the stirring unit in the fermenter, for example, and the flow of culture medium which this causes. In some exemplified embodiments, the holder of the exchange unit comprises a cage which surrounds the bundle or bundles of hollow fibres. Additionally, the cage may comprise sufficient large openings so that there is an adequate flow of culture medium against the hollow fibres in the interior. Additionally, in some embodiments, the larger the opening of the cage, the higher the packing density of the hollow fibres in the interior of the cage can be.

In some exemplified embodiments, the exchange unit may be constructed with hollow fibres in a cylindrical shape (cylinder). Additionally, according to the various embodiments, shapes other than cylindrical are also possible in the exchange unit. In some embodiments, in the exchange unit the hollow fibres may run basically in a straight line and connect two opposing sides of the cage essentially in a line. According to embodiments of the instant disclosure, the filling degree of the exchange unit (the packing density of hollow fibres in a ratio to the volume of the exchange unit) may be determined by the ratio of the cross-sectional area occupied by hollow fibres to the total available cross-sectional area over which the hollow fibres can be distributed. In some embodiments, a degree of filling of about 0.19 to 0.50 is utilized. In some exemplified embodiments, the hollow fibres in the exchange unit may be substantially uniformly distributed. In some embodiments of the instant disclosure, whereby hollow fibres of the exchange unit are bundled in a cylindrical shape, the ratio of the cross-sectional area occupied by hollow fibres to the total cross-sectional area of the cylinder in which the hollow fibres are distributed may be 0.19 to 0.50 in the exchange unit. In some embodiments, a degree of filling of approximately 0.35 is exemplified.

In a particular embodiment the exchange unit may comprise a wall with equally distributed openings or a cage as the outer boundary. The culture medium can flow around the hollow fibres through the openings. In some embodiments comprising a cylindrical design of the exchange unit the ratio of the free area of the cylinder surface to the total surface is in a range of 0.2 to 0.6. In some embodiments, a value of about 0.3 is utilized.

According to the instant disclosure, mass transfer may take place along the membrane surface in the exchange unit during the fermentation process in which undesired end products of metabolism or metabolic end products that inhibit growth or are toxic in high concentrations pass through the membrane into the dialysis medium. A suitable ratio, according to the instant disclosure, of the membrane surface in the exchange unit to the volume of the culture medium which is in contact with the exchange unit, may be selected in order that mass transfer can take place in a sufficient amount and at a sufficiently high rate. According to embodiments of the disclosure comprising a semipermeable membrane having a molecular cut-off of more than 15 kDa to 50 kDa in a dialysis fermenter, the corresponding ratio [surface (MK)/vol.(K)] of the membrane surface [surface (MK)] facing the cell-containing culture medium to the volume of the culture medium [vol. (K)] is, in a particular embodiment, in the range of 0.1 cm⁻¹ to 1.3 cm⁻¹ and in another particular embodiment in the range of 0.2 cm⁻¹ to 1.0 cm⁻¹. These values of the ratio may be utilized, according to the instant disclosure, for volumes of culture medium of the order of magnitude of 10 L, 100 L, 1000 L and 2000 L for dialysis fermenters, for example, where each of the said values (x) defines a range with the limits 0.5(x)≦×≦1.5(x) and where the limits are included in the range.

According to some embodiments of the disclosure, the exchange unit may form the connection between the culture medium in the first compartment and the dialysis medium (nutrient solution) in the second compartment. The nutrient solution may be continuously circulated by moving it by means of a pump through a first fluidic connection, for example, through a rigid pipe or a flexible tube, from the second compartment through the hollow fibres of the exchange unit and from there through a further fluidic connection back again into the second compartment. According to the instant disclosure, the term “dialysis flow” refers to the volume of dialysis medium which is pumped per unit of time by the exchange unit. According to the instant disclosure, a surprising and unexpected approximate 140%-160% higher pressure-dependent dialysis flow was discovered for dialysis fermenters according to the disclosure (comparison is based on fermenter embodiments having approximately a 100 L cell-containing culture medium and compared with a known fermenter using a Cuprophan® RC55 8/200 hydrophilic capillary membrane having a wall thickness of 8.4 μm±1.1 μm and an inner diameter of 200 μm±15 μm as well as a cut-off of about 12 kDa). A value measured as an example for a known fermenter using a Cuprophan® RC55 8/200 hydrophilic capillary membrane (described above) was 7 ml/hPa, whereas an exemplary fermenter according to the instant disclosure provided herein was 11 ml/hPa. Additionally, for dialysis fermenters containing approximately a 1000 L cell-containing culture medium, values for dialysis flow and pressure-dependent dialysis flow for a known fermenter using a Cuprophan® RC55 8/200 hydrophilic capillary membrane (described above) were about 9 L/min, 41 ml/hPa versus a device according to the instant disclosure which demonstrated values of 11.5 L/min, 57.5 ml/hPa.

Depending on various parameters, for example the type of cultured cells, the culture conditions, the age and physiological status of the cell culture, the content of dead cells or cell debris and cell components in the culture medium, or the content of high molecular weight metabolic products in the culture medium, aggregates can sometimes be formed of which some can block the pores of the dialysis membrane. This can take place especially with those aggregates whose total size corresponds approximately to, or is smaller than, the size of the pores in the membrane. Blocking of membranes during the process of dialysis fermentation is also referred to as “fouling”.

Fouling can, on the one hand, occur on the side of the dialysis membrane facing the culture medium. It has proven to be particularly advantageous to specifically reduce the porosity of the dialysis membrane on the side facing the culture medium.

Porosity is a physical quantity and represents the ratio of the volume of void space to the total volume of the membrane material. It can be expressed as the ratio [V_{H}/V] of void space volume [V_{H}] to total volume [V=V_{H}+V_{F}] where [V_{F}] is the pure volume of the solid of the membrane material. Typically and also in the following porosity is stated as a percentage.

In a particular embodiment the cross-section of the semipermeable membrane consists of a first and a second layer where the porosity of the first layer is 30% to 40% and the porosity of the second layer is 70% to 80%. According to such embodiments, both layers may directly adjoin one another and the transition between the two layers can be discrete or continuous i.e. in the latter case without a discrete transition. The width of the first layer may be, according to a particular embodiment, 1% to 30% of the total width of the membrane cross-section. In another exemplary embodiment, the first layer faces the culture medium and the second layer faces the dialysis medium. Such an embodiment can reduce or prevent fouling on the side of the dialysis membrane facing the cell-containing culture medium during the operation of the dialysis fermenter. In another embodiment, the arrangement of the different porous layers may be reversed, i.e. the first layer in this case faces the dialysis medium and the second layer faces the culture medium.

Fouling of the dialysis membrane on the side facing the dialysis medium can occur when certain substances passing out of the cell culture medium into the nutrient solution exceed threshold concentrations in the nutrient medium. These substances can form aggregates or precipitates when threshold concentrations are exceeded depending on the respective cell culture, its culture medium, the composition of the nutrient medium, the physiological status of the culture, the metabolic products that are formed and the cell components that are released. If these insoluble components enter the hollow fibres their conducting cross-section can be completely blocked.

If small aggregates enter the hollow fibres, they can close the membrane pores and thus impede the mass transfer. An undesired effect of this is that as the fouling increases, the pump has to build up an ever increasing pressure for the inflow into the exchange unit to ensure a constant flow rate. This effect can lead to a cessation of the flow through the exchange unit. In this case metabolic end products are no longer transported out of the culture medium and the cell culture can no longer be supplied and maintained with nutrients by means of dialysis.

In order to avoid such disadvantages, it is very advantageous to connect the inlet lead of the exchange unit to a functional unit which is suitable for preventing precipitates that are formed in the dialysis medium from entering the exchange unit through the inlet. This functional unit can consist of a sieve, a filter, a continuous flow centrifuge or a separation device for sedimenting or floating the precipitate. The said functional unit is placed in front (upstream) of the inlet into the exchange unit on the side of the dialysis medium.

The effects of fouling as described above typically become noticeable after 9 to 10 days. This time period may be different and even be longer depending on the cultured cell line. Thus the reduction and/or prevention of fouling based on the membrane described according to the instant disclosure is a technical advantages with regard to dialysis flow and pressure of the instant application.

In some embodiments of the instant disclosure, a filter is provided as a functional unit such that it is suitable for preventing precipitates formed in the nutrient solution from entering the exchange unit through the inlet. An exemplified embodiment of the instant disclosure includes a filter comprising a pore size of 2 μm to 0.02 μm, where 2 μm and 0.02 μm are included in the range. A pore size is 0.5 μm±5% also comprises some embodiments of the instant disclosure. A filter, according to embodiments of the instant disclosure, does not limit the dialysis flow and the pressure-dependent dialysis flow through the exchange unit.

Embodiments of the instant disclosure provided and disclosed herein allow for the period of use of the exchange unit to be considerably increased by preventing insoluble components in the dialysis medium from coming into contact with the membranes of the hollow fibres in the exchange unit. The duration of the fermentation process, according to the instant disclosure, may also be increased in this manner. Thus, it is possible for example, by way of embodiments of the instant disclosure to achieve longer production times of target molecules in cultures in a stationary phase. Examples of target molecules may include antibodies and other proteins which are secreted from the interior of cells into the surrounding medium, for example.

According to further embodiments of the instant disclosure, a process for culturing cells is provided. According to some embodiments, a cell culture is kept in substantially homogeneous suspension in a culture medium by means of a stirring device under controlled environmental conditions in a biorector (e.g., a dialysis fermenter). Nutrients for the cells may be fed in and waste products of the cells may be discharged, wherein a dialysis medium (e.g., a nutrient solution) that is separate from the culture medium may be used which flows in a flow path, which is separated from the culture medium by a semipermeable membrane where the membrane is designed such that it is permeable to the nutrients and the waste products of the cells but is impermeable to higher molecular components of the culture medium, and wherein the culture medium containing the cells is led past one side of the membrane and the dialysis medium containing the nutrients is led past the other side of the membrane such that nutrients from the dialysis medium can pass through the membrane into the culture medium and waste products from the culture medium can pass into the dialysis medium. According to some embodiments, the molecular cut-off of the membrane may be in the range of about 15 kDa to 50 kDa, where 15 kDa and 50 kDa are included in the range.

The fermentation process according to the instant disclosure may be utilized for culturing all species and types of biological cells which can be kept in suspension culture. For example, insect cells and mammalian cells, as well as cell lines derived from mammalian cells, may be utilized. Hybridoma cells may also be utilized, including hybridoma cells which secrete monoclonal antibodies. The process according to the disclosure particularly takes place in a dialysis fermenter.

Further, according to the instant disclosure a synthetic material (such as a semipermeable membrane) in the exchange unit may be used as opposed a known cellulose-based membranes (such as Cuprophan®). An example synthetic material according to the instant disclosure includes polyethersulfone (PES). Alternative synthetic materials as described herein may also be used and achieve comparable, surprisingly and unexpected advantageous results.

Additionally, the instant disclosure is further advantageous in that it cellulose-based hollow fibres are no longer available in a suitable form on the world market. (whereas synthetic materials as disclosed herein, including P ES-based hollow fibres are available worldwide. Additionally, embodiments of the instant disclosure allow for preparing hollow fibre modules containing synthetic hollow fibres (before carrying out fermentation) in a substantially shorter time frame. Further, unlike cellulose-based hollow fibres which have to be immersed in a preservative solution for storage (this solution is known to contain substances which are not compatible with the culture of eukaryotic cells), embodiments of the instant disclosure do not require this preservative solution. Thus, unlike with other known cellulose-based hollow fibres, embodiments of the instant disclosure do not therefore require the preservative solution to be removed by washing (a process which is cumbersome and increases time and materials required to prepare fermentation processes). Depending on the hollow fibre module, one expects a time requirement of about three hours per module and fermentation. In contrast, no pretreatment is necessary for embodiments of the instant disclosure, comprising polyethersulfone-based modules for example. The modules can be stored without a preservative and directly incorporated in the fermenter and used as intended.

Further, unlike cellulose-based fibres which shrink when they are dried (thus if re-use is intended, re-immersion within a stabilization solution and heat-sterilization after fermentation and subsequent cleaning are completed are required, processes which are very laborious) embodiments of the instant disclosure (for example, P ES-based hollow fibre modules) can be dried in the air at room temperature after cleaning and can be stored in a dry state at room temperature.

Additionally, embodiments of disclosure provided herein (for example, PES-based hollow fibre modules) in general comprise a higher pressure-dependent dialysis flow. This allows the pump in the dialysis feed line to have a cheaper design because the pump capacity can be down-scaled.

### Illustrative Embodiments

The following is a list of illustrative embodiments according to the instant disclosure which represent various embodiments of the instant disclosure.

1. A method of using a semipermeable membrane having a molecular cut-off of more than 15 kDa to 50 kDa, where 15 kDa and 50 kDa are included in the range, in a dialysis fermenter as a separation layer between (a) the cell-containing liquid culture medium and (b) the non-cell-containing dialysis medium (nutrient solution).

2. The use according to 1, wherein the molecular cut-off of the semipermeable membrane is in a range of 20 kDa to 40 kDa, where 25 kDa and 40 kDa are included in the range.

3. The use according to one of 1 and 2, wherein the molecular cut-off of the semipermeable membrane is 35 kDa±5%.

4. The use according to any of 1 to 3, wherein the material of the membrane is selected from the group comprising regenerated cellulose, modified cellulose, polysulfone (PSU), polyacrylonitrile (PAN), polymethylmethacrylate (PMMA), polyvinyl alcohol (PVA) and polyarylethersulfone (PAES).

5. The use according to 4, wherein the material of the membrane is polysulfone and particularly polyethersulfone.

6. The use according to any one of 1 to 5, wherein the semipermeable membrane consists of a first and a second layer.

7. The use according to 6, wherein the porosity of the first layer is 30% to 40% and that of the second layer is 70% to 80%.

8. The use according to 7, wherein the first and the second layer are directly adjacent and the thickness of the first layer is 1% to 30% of the cross-section of the membrane.

9. The use according to 8, wherein in the exchange unit the first layer of the membrane faces the cell-containing culture medium.

10. A dialysis fermenter comprising a first compartment containing a cell-containing liquid culture medium and a second compartment containing a non-cell-containing dialysis medium (e.g., a nutrient solution), further comprising an exchange unit (e.g., a dialyzer) with a semipermeable membrane which dips into the culture medium, wherein the exchange unit (e.g., the filter module) is fluidically connected to the second compartment by an inlet as well as an outlet, further comprising a pump which feeds dialysis medium from the second compartment into the exchange unit and from there back again into the second compartment, wherein mass transfer takes place between the culture medium and the dialysis medium along the semipermeable membrane by means of diffusion and/or ultrafiltration, wherein the molecular cut-off of the semipermeable membrane is in a range of about 15 kDa to about 50 kDa, where 15 kDa and 50 kDa are included in the range.

11. The dialysis fermenter according to 10, wherein the first compartment additionally contains a stirring unit, wherein the stirring unit is suitable for keeping the cells in suspension and particularly in an essentially homogeneous suspension.

12. The dialysis fermenter according to any of 10 and 11, wherein the membrane in the exchange unit is present as a plurality of hollow fibres arranged in parallel.

13. The dialysis fermenter according to any of 10 to 12, wherein the inlet to the exchange unit additionally comprises a functional unit which is suitable for preventing precipitates that are formed in the dialysis medium from entering the hollow fibres of the exchange unit through the inlet.

14. The dialysis fermenter according to 13, wherein the functional unit is selected from the group comprising a sieve, a filter, a centrifuge and a separation device for sedimenting or floating the precipitate.

15. The dialysis fermenter according to 14, wherein the functional unit is a filter.

16. The dialysis fermenter according to 15, wherein the pore size of the filter is approximately 2 μm to approximately 0.02 μm, where 2 μm and 0.02 μm are included in the range.

17. The dialysis fermenter according to 16, wherein the pore size of the filter is approximately 0.5 μm±5%.

18. The dialysis fermenter according to any of 10 to 17, wherein in the exchange unit the ratio [surf. (MK)/vol.(K)] of the membrane surface area [surf. (MK)] facing the cell-containing culture medium to the volume of the culture medium [vol.(K)] is in the range of 0.1 cm⁻¹ to 1.3 cm⁻¹, where 0.1 cm⁻¹ and 1.3 cm⁻¹ are included in the range.

19. The dialysis fermenter according to 18, wherein in the exchange unit the ratio [surf. (MK)/vol.(K)] of the membrane surface area [surf. (MK)] facing the cell-containing culture medium to the volume of the culture medium [vol.(K)] is in the range of 0.2 cm⁻¹ to 1.0 cm⁻¹, where 0.2 cm⁻¹ and 1.0 cm⁻¹ are included in the range.

20. The dialysis fermenter according to any of 12 to 19, wherein the average inner diameter of the hollow fibres in the exchange unit has a value in the range of approximately 50 μm to approximately 500 μm, where 50 μm and 500 μm are included in the range.

21. The dialysis fermenter according to any of 12 to 20, wherein the average outer diameter of the hollow fibres in the exchange unit has a value in the range of approximately 65 μm to approximately 600 μm, where 65 μm and 600 μm are included in the range.

22. The dialysis fermenter according to any of 20 and 21, wherein in the exchange unit the average outer diameter of the hollow fibres is 260 μm±10% and the average inner diameter of the hollow fibres is 190 μm±10%.

23. The dialysis fermenter according to any of 10 to 22, wherein the exchange unit has a cylindrical shape and in the exchange unit the ratio of the cross-sectional area occupied by hollow fibres to the total cross-sectional area of the cylinder over which the hollow fibres are distributed is approximately 0.19 to 0.50, where 0.19 and 0.50 are included in the range.

24. The dialysis fermenter according to 23, wherein the exchange unit contains openings which are suitable for allowing the culture medium to flow around the hollow fibres and wherein the ratio of the area of the cylinder surface kept open to the total surface of the cylinder is in a range of approximately 0.2 to 0.6, where 0.2 and 0.6 are included in the range.

25. An operation of a dialysis fermenter by employing a use according to one of 1 to 9.

26. Carrying out a dialysis fermentation employing a use according to one of 1 to 9.

27. Carrying out a dialysis fermentation by means of a dialysis fermenter according to one of 10 to 24.

28. Preparing a dialysis fermenter according to any one of 10 to 24 employing a use according to one of 1 to 9, as well as the result of this preparation.

29. A process for culturing cells using the provided results of 28 or of a dialysis fermenter according to one of 10 to 24.

30. A process for culturing cells in which a cell culture is kept in substantially homogeneous suspension in a culture medium by means of a stirring device under controlled environmental conditions in a biorector (e.g., a dialysis fermenter), nutrients for the cells are fed in and waste products of the cells are discharged, wherein a dialysis medium (e.g., a nutrient solution) that is separate from the culture medium is used which flows in a flow path which is separated from the culture medium by a semipermeable membrane where the membrane is designed such that it is permeable to the nutrients and the waste products of the cells but is impermeable to higher molecular components of the culture medium and wherein the culture medium containing the cells is led past one side of the membrane and the dialysis medium containing the nutrients is led past the other side of the membrane such that nutrients from the dialysis medium can pass through the membrane into the culture medium and waste products from the culture medium can pass into the dialysis medium, characterized in that the molecular cut-off of the membrane is in the range of approximately 15 kDa to 50 kDa, where 15 kDa and 50 kDa are included in the range.

31. The process according to 30, wherein the biorector is a dialysis fermenter according to one of 10 to 24 or a dialysis fermenter as a result of 28.

32. The process according to one of 30 and 31, wherein the cells are suitable for being kept in a suspension culture.

33. The process according to 32, wherein the cells are selected from the group comprising insect cells, mammalian cells and cell lines derived from these cells.

34. The process according to 33, wherein the cells are selected from the group consisting of fusion products, for example hybridoma cells, and transformed cells.

35. The process according to one of 30 to 34, wherein the cells produce one or more desired substance(s) and optionally secrete them into the culture medium.

36. The process according to 35, wherein the molecular weight(s) of the desired substance(s) is/are larger than the molecular cut-off of the membrane (i.e. is larger than a value in the range of 15 kDa to 50 kDa, where 15 kDa and 50 kDa are included in the range).

37. The process according to 36, wherein the desired substance(s) is/are selected from the group comprising an immunoglobulin, a coagulation factor, a growth factor, a precursor of a signal molecule, for example a peptide hormone, an enzyme, a subunit of a protein complex and fragments or derivatives thereof.

38. The process according to one of 30 to 37, wherein the dialysis medium firstly passes through through a functional unit which is suitable for preventing precipitates, if present in the dialysis medium, before it passes through the membrane into the culture medium.

The following examples and figures are provided for the purpose of demonstrating various embodiments of the instant disclosure and aiding in an understanding of the present disclosure, the true scope of which is set forth in the appended claims. These examples are not intended to, and should not be construed as, limiting the scope or spirit of the instant disclosure in any way. It should also be understood that modifications can be made in the procedures set forth without departing from the spirit of the disclosure.

### EXAMPLES

### Example 1

### Comparative Dialysis Fermentation of a Mouse Hybridoma Cell Line

Hybridoma cell line A produces a monoclonal antibody and secretes it into the surrounding liquid culture medium. The antibody, as the product, can be qualitatively and quantitatively detected in the culture medium by using standard methods. The cell line was cultured according to an embodiment of instant disclosure, using a semipermeable membrane having a molecular cut-off of 15 kDa to 50 kDa, where 15 kDa and 50 kDa are included in the range, in a dialysis fermenter as a separation layer between (a) the cell-containing liquid culture medium and (b) the non-cell-containing dialysis medium (nutrient solution).

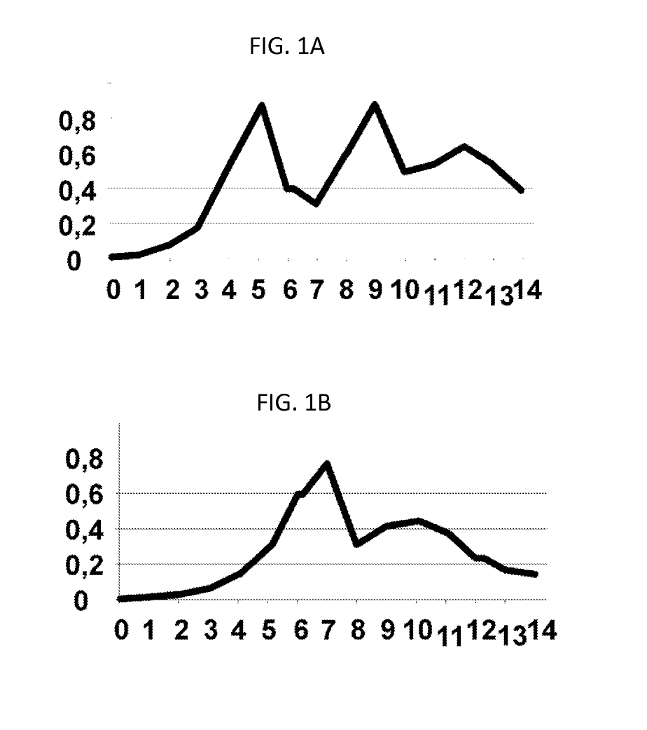

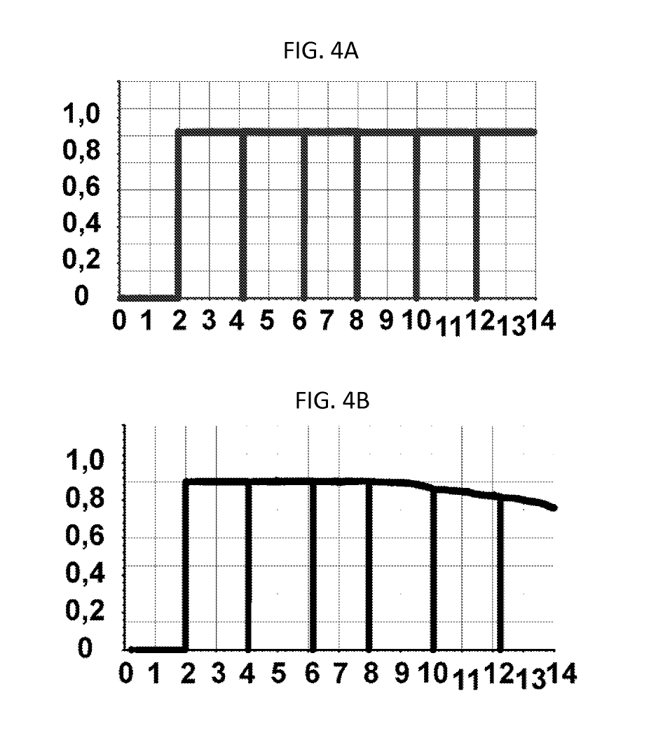

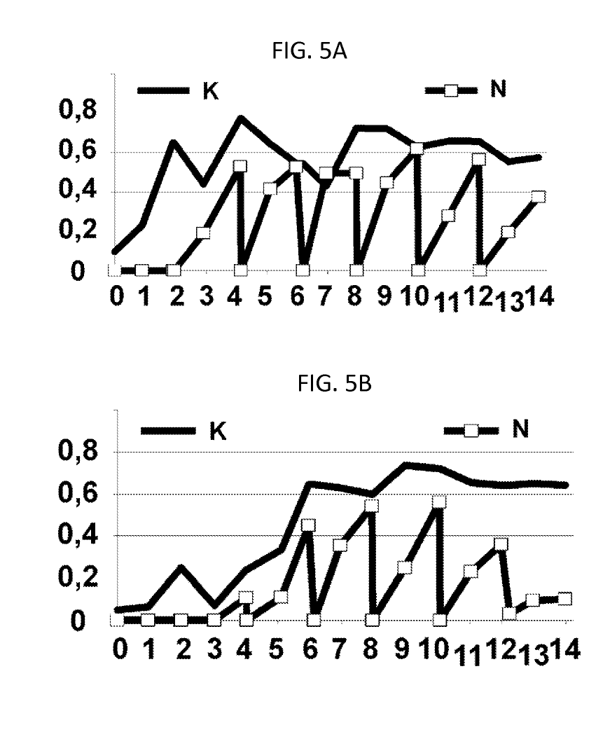

With reference to FIGS. 1 to 5, the course of the fermentation 1 using the mouse hybridoma cell line A is compared with the course of the fermentation 2 using the same hybridoma cell line A in various diagrams. Each figure shows the measured results for a different parameter. Each diagram denoted “A” corresponds to results from fermentation 1; diagrams labelled “B” are those which show the data from fermentation 2.

A principal technical difference between fermentation 1 and fermentation 2 is that in the dialysis fermenter in the case of fermentation 1 the inflow line of the exchange unit is equipped with a functional unit which is suitable for preventing precipitates that are formed in the nutrient solution from entering the exchange unit through the inlet. Otherwise in fermentation 1 and 2 the same exchange units according to the disclosure were used. These contained PES-based hollow fibres having a molecular cut-off of the semipermeable membrane in a range of about 35 kDa.

The time courses of the live cell density, of the product concentration in the cell-containing culture medium, of the module input pressure (pressure in the inlet to the exchange unit), of the dialysis flow and of the lactate concentration in the culture medium and in the nutrient solution are shown. The dialysis medium was exchanged i.e., replaced by fresh medium, in each case after 2, 4, 6, 8, 10 and 12 days. The composition of the dialysis medium was not changed during the processes shown. Both fermentation processes were each terminated after 14 days.

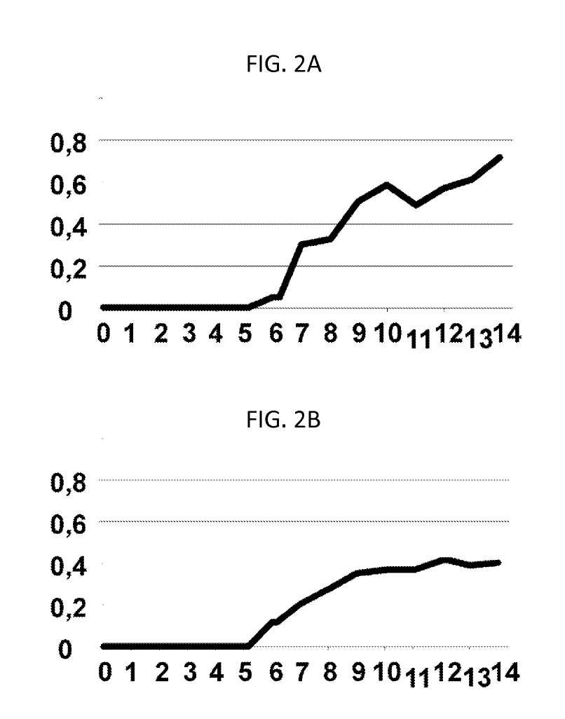

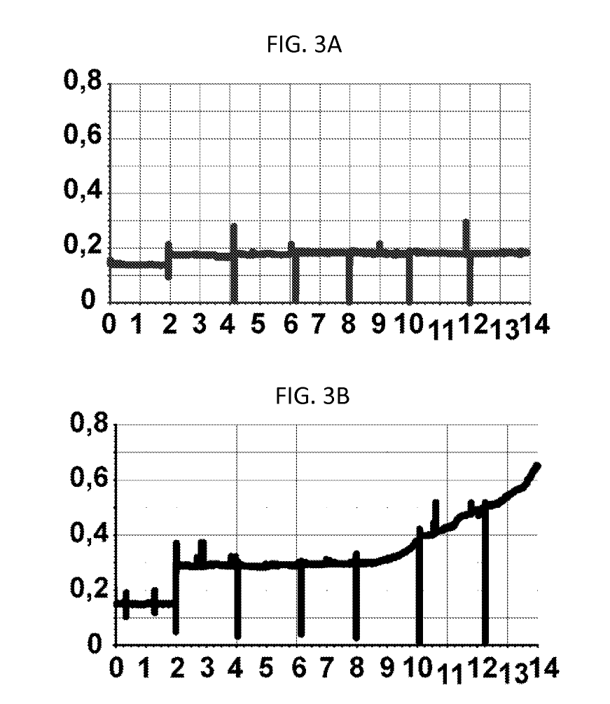

The live cell density decreased in fermentation 2 towards the end of the process compared to fermentation 1 (FIG. 1). Also, the product concentration in the dialysis fermenter no longer significantly increased after fermentation day 9 in fermentation 2. By contrast, in fermentation 1 the product concentration continued to increase up to day 14 (FIG. 2). After about day 9 the inlet pressure on the exchange unit increased continuously in fermentation 2 (FIG. 3) whereas the dialysis flow decreased in fermentation 2.

The time course of the lactate concentration in the culture medium and in the nutrient solution was determined as an example of a measure for the mass transfer across the exchange unit. The nutrient solution was replaced in each case after 2 days in the fermentation preparations 1 and 2. This resulted in a continuous increase in the lactate concentration in the nutrient solution until after 2 days the lactate concentration was almost equal in the culture medium and in the nutrient solution. Since mass transfer through the exchange unit was driven especially by the concentration difference of a given substance through the semipermeable membrane, the nutrient solution was exchanged at this time. In the case of fermentation 2 the concentration time courses of lactate in the culture medium and in the nutrient solution showed that the lactate concentration in the nutrient solution remained in each case below the lactate concentration in the culture medium towards the end of the fermentation, after about day 11. In the case of fermentation 1 the lactate concentration in the nutrient solution reached approximately the lactate concentration in the culture medium even towards the end of the fermentation. This effect was particularly unexpected and surprising, and was interpreted to mean that the exchange of lactate by the exchange unit is much better in the case of fermentation 1 than with fermentation 2. An examination showed that in the case of fermentation 2 the exchange unit was blocked by a precipitate. This precipitate had formed in the nutrient solution. The precipitate could also be observed macroscopically in the supply container containing the nutrient solution.

All publications, patents and applications are hereby incorporated by reference in their entirety to the same extent as if each such reference was specifically and individually indicated to be incorporated by reference in its entirety.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within the known or customary practice in the art to which this disclosure pertains.

What is claimed is:

1. A dialysis fermenter, comprising:
a first compartment adapted for containing a cell-containing liquid culture medium;
a second compartment adapted for containing a nutrient solution;
an exchange unit having a semipermeable membrane and a plurality of hollow fibres, the exchange unit dipping into the first compartment such that when the first compartment contains liquid culture medium the exchange unit is capable of contacting the liquid culture medium, the exchange unit being fluidically connected to the second compartment by an inlet as well as an outlet, the inlet having a functional unit adapted for preventing precipitates within the nutrient solution from entering the hollow fibres through the inlet;
a pump configured to feed the nutrient solution from the second compartment into the exchange unit and from the exchange unit into the second compartment, wherein mass transfer takes place between the liquid culture medium and the nutrient solution along the semipermeable membrane by means of one of diffusion and ultrafiltration, the semipermeable membrane having a molecular cut-off in a range of approximately 15 kDa to approximately 50 kDa, 15 kDa and 50 kDa being included in the range.

2. The dialysis fermenter of claim 1, wherein the first compartment comprises a stirring unit adapted for keeping the cells in the cell-containing liquid culture medium in suspension.

3. The dialysis fermenter of claim 1, wherein the plurality of hollow fibres of the semipermeable membrane are arranged in parallel.

4. The dialysis fermenter of claim 1, wherein the exchange unit comprises a ratio of the semipermeable membrane surface area facing the cell-containing culture medium to the liquid culture medium volume in the range of approximately 0.1 cm⁻¹ to 1.3 cm⁻¹, 0.1 cm⁻¹ and 1.3 cm⁻¹ being included within the range.

5. The dialysis fermenter of claim 1, wherein the exchange unit has a cylindrical shape.

6. The dialysis fermenter of claim 5, wherein the exchange unit contains openings which are suitable for allowing the liquid culture medium to flow around the hollow fibres.

7. A method of using a semipermeable membrane having a molecular cut-off of approximately 15 kDa to 50 kDA, 15 kDa and 50 kDa being included in the range, in a dialysis fermenter according to claim 1, the semipermeable membrane comprising a separation layer between the cell-containing liquid culture medium and the nutrient solution.

8. The method of claim 7, wherein the material of the membrane is selected from the group comprising regenerated cellulose, modified cellulose, polysulfone (PSU), polyacrylonitrile (PAN), polymethylmethacrylate (PMMA), polyvinyl alcohol (PVA) and polyarylethersulfone (PAES).

9. A process for culturing cells comprising the steps of:
maintaining a cell culture in substantially homogeneous suspension in a culture medium by means of a stirring device under controlled environmental conditions in a bioreactor;
feeding nutrients for the cells into the cell culture medium; and
discharging waste products of the cells, wherein a dialysis medium that is separate from the culture medium is used which flows in a flow path which is separated from the culture medium by a semipermeable membrane where the membrane is designed such that it is permeable to the nutrients and the waste products of the cells but is impermeable to higher molecular components of the culture medium and wherein the culture medium containing the cells is led past one side of the membrane and the dialysis medium containing the nutrients is led past the other side of the membrane such that nutrients from the dialysis medium firstly pass through a functional unit which is suitable for preventing precipitates, if present in the dialysis medium, and secondly pass through the membrane into the culture medium and waste products from the culture medium pass into the dialysis medium, wherein the molecular cut-off of the membrane is in the range of 15 kDa to 50 kDa, 15 kDa and 50 kDa being included within the range.

10. The process of claim 9, wherein the cells are suitable for being kept in a suspension culture.

11. The process of claim 9, wherein the cells secrete a desired substance into the culture medium.

12. The process of claim 11, wherein the desired substance has a molecular weight which is larger than the molecular cut-off of the semipermeable membrane.

13. The process of claim 12, wherein the desired substance is selected from the group comprising an immunoglobulin, a coagulation factor, a growth factor, a precursor of a signal molecule, an enzyme, a subunit of a protein complex, and fragments or derivatives thereof.

14. The process of claim 9, wherein the cells are selected from the group comprising insect cells, mammalian cells, and cell lines derived from these cells.

15. The process of claim 9, wherein the cells are selected from the group comprising hybridoma cells and transformed cells.
